(19) United States
(12) Patent Application Publication
(10) Pub. No.: US 2022/0403340 A1
(43) Pub. Date: Dec. 22, 2022

(54) MEDIA FOR CRYOPRESERVED CELLS AND METHODS OF MAKING AND USING SAME

(71) Applicant: Washington University, St. Louis, MO (US)

(72) Inventors: Jeffrey Millman, St. Louis, MO (US); Leonardo Velazco-Cruz, St. Louis, MO (US); Punn Augsornworawat, St. Louis, MO (US)

(21) Appl. No.: 17/839,138

(22) Filed: Jun. 13, 2022

Related U.S. Application Data
(60) Provisional application No. 63/209,435, filed on Jun. 11, 2021.

(51) Int. Cl. C12N 5/071; A01N 1/02

(52) U.S. Cl. CPC C12N 5/0676; A01N 1/021; C12N 2501/73; C12N 2500/32; C12N 2500/34; C12N 2500/22

(57) ABSTRACT

Among the various aspects of the present disclosure is the provision of a media for cryopreserved cells and methods of making and using same. An aspect of the present disclosure provides for a cell media formulation comprising (e.g., for day 0) one or more components selected from: MCDB 131; Glutamax; P/S; BSA; Glucose; ZnSO4; an enzyme for digesting DNA (e.g., DNASE1); and/or an apoptosis inhibitor (e.g., BI-6C9).

### CROSS REFERENCE TO RELATED APPLICATIONS

This application claims the benefit of U.S. Provisional Application 63/209,435, filed Jun. 11, 2021 the disclosure of which is hereby incorporated by reference in its entirety.

### FIELD OF THE TECHNOLOGY

This disclosure generally relates to methods, compositions, kits, and agents useful for cryopreserved cells.

### BACKGROUND

Cryopreservation is an enabling technology providing on-demand access and distribution of biological material (e.g., cells and tissues). It was estimated that in 2019 there were 463 million people with diabetes worldwide and this number is expected to increase to 700 million by 2045. Elucidating the mechanisms underlying diabetes is hampered by limitations in the availability and accessibility of human islet cells for single-cell characterization studies. Native islets are multi-cellular structures, which makes them more challenging to cryopreserve than single cells because of non-uniform water and cryoprotectant content in the core cells compared to cells on the outer layer during freezing. Thus, an optimized procedure for the cryopreservation of dissociated islet cells may be beneficial in diabetes research and in improving transplantation outcomes.

Thus, there is a need in the art compositions and methods for thawing cryopreserved cells, for example human pluripotent stem cell-derived islets (SC-islets), that are functionally similar to pancreatic islet beta cells, and used in regenerative medicine.

### SUMMARY

Among the various aspects of the present disclosure are provided compositions and methods for thawing, maintaining, and/or re-aggregating cell therapies.

In some embodiments, the present disclosure provides a re-aggregation medium comprising a basal medium, at least one glutamine alternative, at least one antibiotic, one or more albumin, at least one energy substrate, at least one trace element, and optionally one or more deoxyribonuclease and at least one apoptosis inhibitor. In certain embodiments, the basal medium comprises trace elements, putrescine, adenine, thymidine and sodium bicarbonate. In an exemplary embodiment, the basal medium is MCDB131. In each of the above embodiments, the glutamine alternative is L-alanyl-L-glutamine. In each of the preceding embodiments, the antibiotic is penicillin and streptomycin. In each of the preceding embodiments, the albumin is bovine serum albumin. In each of the preceding embodiments, the energy substrate is glucose. In each of the preceding embodiments, the trace elements comprise ZnSO₄. In each of the preceding embodiments, when present, the deoxyribonuclease is DNase I and the apoptosis inhibitor is BI-6C9.

According to the present disclosure is provided methods of thawing, resizing, maintaining, or passaging at least one SC-islet cell by culturing the at least one SC-islet cell in the presence of basal medium, at least one glutamine alternative, at least one antibiotic, one or more albumin, at least one energy substrate, at least one trace element, and optionally one or more deoxyribonuclease and at least one apoptosis inhibitor. In certain embodiments, the basal medium comprises trace elements, putrescine, adenine, thymidine and sodium bicarbonate. In an exemplary embodiment, the basal medium is MCDB131. In each of the above embodiments, the glutamine alternative is L-alanyl-L-glutamine. In each of the preceding embodiments, the antibiotic is penicillin and streptomycin. In each of the preceding embodiments, the albumin is bovine serum albumin. In each of the preceding embodiments, the energy substrate is glucose. In each of the preceding embodiments, the trace elements comprise ZnSO4. In each of the preceding embodiments, when present, the deoxyribonuclease is DNase I and the apoptosis inhibitor is BI-6C9.

In some embodiments, the SC-islet cells were previously cryopreserved and then thawed by, in part, contacting the SC-islet cell with a first re-aggregation medium comprising basal medium, at least one glutamine alternative, at least one antibiotic, one or more albumin, at least one energy substrate, at least one trace element, one or more deoxyribonuclease and at least one apoptosis inhibitor for about 24 hours. In some embodiments, the method further comprises contacting the SC-islet cell with a second re-aggregation medium comprising basal medium, at least one glutamine alternative, at least one antibiotic, one or more albumin, at least one energy substrate, at least one trace element.

In each of the above embodiments, contacting the SC-islet with medium results in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

In some embodiments, the thawed SC-islets are incubated for a period of time sufficient to result in: high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

In some embodiments, contacting the thawed SC-islets in one or both of the formulations results in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

Another aspect of the present disclosure encompasses kits for preparing the re-aggregation media of the disclosure wherein the kits include individually packaged media components (e.g. at least one glutamine alternative, at least one antibiotic, one or more albumin, at least one energy substrate, at least one trace element, and optionally one or more deoxyribonuclease and at least one apoptosis inhibitor), basal medium, and instructions for preparing the medium.

### BRIEF DESCRIPTION OF THE FIGURES

The patent or application file contains at least one drawing executed in color. Copies of this patent or application publication with color drawing(s) will be provided by the Office upon request and payment of the necessary fee.

FIG. 1 shows conventional media causes undesirable cell clumping of SC islets after thaw from cryopreservation.

FIG. 2A-2B show exemplary media formulations according to the present disclosure improve cell recovery and clustering morphology. FIG. 2A shows cell clumping of SC islets after thaw from cryopreservation in control S6 media. FIG. 2B shows improved cell morphology and function of SC islets after thaw from cryopreservation using media formulations of Table 1 and Table 2.

FIG. 3A-3C show exemplary media of the disclosure provides improved gene expression for SC Islets and maintains Beta cell identity. FIG. 3A shows the relative expression of INS and MAFA in standard control M6 media versus new media formulations of Table 1 and Table 2. FIG. 3B shows SC islets maintain insulin expression after thaw in the media formulations of the disclosure. FIG. 3C shows SC islet cells maintain C-peptide and NKX6-1 in media formulations of the disclosure.

### DETAILED DESCRIPTION

The present disclosure is based, at least in part, on the discovery that specific formulations of medium which provides improved thawing of cryopreserved cells. In particular, the present disclosure provides an effective protocol for thawing cryopreserved cells (e.g., SC-β cells). In certain embodiments, the protocol for thawing cryopreserved SC-β cells results in thawed SC-β cells which are functionally similar to pancreatic islet beta cells, and are useful in regenerative medicine, for example, to address type-1 diabetes. The present disclosure provides a composition of matter (i.e., a thawing media and a culture media) that significantly improves cell survival, growth, and differentiation relative to standard techniques. In addition, the present disclosure relates to methods of using cells thawed according to the protocol disclosed herein.

Human pluripotent stem cell-derived islets (SC-islets) secrete insulin and have value in diabetes cell replacement therapy. However, there is a problem thawing cryopreserved SC-islets that leads to significant cell death after several days in culture. Moreover, standard thawing techniques result in unfavorable cluster morphologies leading to poor islet health and function. Resizing SC-islets leads to improvements in SC-islets. However, this also results in the death of some cells.

As shown herein, successful thawing of SC-islets improves their utility for diabetes cell replacement therapy. The present disclosure, inter alia, provides a re-aggregation media formulation allowing for high recovery yields of cryopreserved SC-islets; media formulations which lead to improved Islet health resulting in improved function; and a re-aggregation media formulation allowing for high recovery yield of resized SC-islets

Maturation of SC-islets produced in vitro is still a limitation for the utility of these cells in cell replacement therapy. The present disclosure provides for improving the maturation of the cells, increasing their utility for diabetes cell replacement therapy.

Additional aspects of the disclosure are described below.

### I. Compositions

Aspects of the present disclosure provide for compositions useful in the thawing of cell therapies after cryopreservation. The term “re-aggregation medium” as used herein refers to a liquid medium which can be used to thaw cell therapies through, in part, a protocol including replacement of a cryopreservation medium with a medium of the disclosure. In another aspect, as used herein a “re-aggregation medium” refers to a liquid medium which useful for preventing cell death after resizing of SC islets.

A “re-aggregation medium” (also referred to herein as a “media” or “medium”) is a medium for thawing and subsequent culture of cells containing nutrients and factors that maintain cell viability, support proliferation, and/or reduces apoptosis. The medium may contain any of the following nutrients in appropriate amounts and combinations: salt(s), buffer(s), amino acids, glucose or other sugar(s), antibiotics, serum or serum replacement, and other components such as, but not limited to, apoptosis inhibitor(s), DNase, cofactors, and trace elements. In some embodiments, the re-aggregation medium is chemically defined medium.

In some embodiments, the re-aggregation medium comprises a basal medium to which one or more supplements are added. As used herein, a “basal medium” is typically an unsupplemented medium (e.g., MCBD 131, Eagle's minimal essential medium (EMEM); Dulbecco's modified Eagle's medium (DMEM)). As will be appreciated by those of skill in the art, a basal medium can comprises a variety of components such as one or more nucleobases and/or nucleosides (e.g. adenine and thymidine), trace elements, polyamine (e.g. putrescine), and sodium bicarbonate. In some embodiments, the basal medium can further comprise amino acids, salts (e.g., calcium chloride, potassium chloride, magnesium sulfate, sodium chloride, monosodium phosphate), sugars (e.g., glucose), vitamins (e.g., folic acid, nicotinamide, riboflavin, B12), iron, pH indicators (e.g., phenol red), proteins (e.g., albumin), hormones (e.g., insulin), glycoproteins (e.g., transferrin), minerals (e.g., selenium), serum (e.g., fetal bovine serum), antibiotics, antimycotics and glycosaminoglycans. In some embodiments, a basal medium provides a balanced electrolyte solution. In some embodiments, a basal medium for use in the compositions herein can be about 10% to about 90% (e.g., about 10%, about 20%, about 30%, about 40%, about 50%, about 60%, about 70%, about 80%, about 90%) of the total volume. In a preferred embodiment, the basal medium is MCDB131.

In some embodiments, re-aggregation medium comprises an essential amino acid alternative. As used herein, an “essential” amino acid refers to an amino acid that cannot be made by the cultured cells. Non-limiting examples of essential amino acids suitable for use herein can include L-Arginine, L-Cysteine, L-Cystine, L-Glutamine, L-Histidine, L-Isoleucine, L-Leucine, L-Lysine, L-Methionine, L-Phenylalanine, L-Threonine, L-Tryptophan, L-Tyrosine, and L-Valine. In some embodiments, the essential amino acid alternative is a glutamine alternative. In a certain embodiments, the glutamine alternative is a L-alanyl-L-glutamine dipeptide. In some, a L-alanyl-L-glutamine dipeptide for use in the compositions herein can be used in a final concentration of about 0.5 to about 10 mM (e.g. about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM). in a preferred embodiment, a re-aggregation medium comprises about 2 mM L-alanyl-L-glutamine dipeptide.

In some embodiments, a re-aggregation medium herein can contain serum, serum replacement medium, or any combination thereof. In some embodiments, serum for use herein can be a complex mix of albumins, growth factors, growth inhibitors, and the like. In some embodiments, serum for use in the compositions herein can be at a concentration of between about 1% to 50% (e.g., about 1%, about 5%, about 10%, about 20%, about 30%, about 40%, about 50%). In some embodiments, a re-aggregation medium comprises a serum component such as albumin. In certain embodiments, the albumin is bovine serum albumin. In some embodiments, the albumin for use in the compositions herein can be used in a final concentration of about 0.01 to about 1 mM (e.g. about 0.01 mM, about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM). In a preferred embodiment, a re-aggregation medium comprises about 300 μM bovine serum albumin.

In some embodiments, a re-aggregation medium herein can have one or more energy substrates. In some embodiments, a re-aggregation medium herein can have one or more energy substrates at a total amount of about 0.001 g/L to about 1 g/L (e.g., about 0.001 g/L, about 0.005 g/L, about 0.01 g/L, about 0.015 g/L, about 0.02 g/L, about 0.025 g/L, about 0.035 g/L, about 0.04 g/L, about 0.045 g/L, about 0.05 g/L, about 0.055 g/L, about 0.06 g/L, about 0.065 g/L, about 0.05 g/L, about 0.07 g/L, about 0.075 g/L, about 0.08 g/L, about 0.085 g/L, about 0.09 g/L, about 0.095 g/L, about 0.1 g/L, about 0.15 g/L, about 0.2 g/L, about 0.3 g/L, about 0.4 g/L, about 0.5 g/L, about 0.6 g/L, about 0.7 g/L, about 0.8 g/L, about 0.9 g/L, about 0.1 g/L). In some embodiments, compositions herein can have one or more energy substrates derived from carbohydrates. Non-limiting examples of carbohydrates suitable for use herein can be glucose, galactose, maltose, fructose, and the like. In a preferred embodiment, a re-aggregation medium comprises about 0.115 g/L glucose.

In some embodiments, a re-aggregation medium herein can have one or more trace elements. Non-limiting examples of trace elements suitable for use herein can include zinc, copper, selenium, tricarboxylic acid intermediates, and the like. In certain embodiments, a trace elements include ZnSO₄.

In some embodiments, a cryopreservation medium herein can have one or more antibiotics. Non-limiting examples of antibiotics suitable for use herein can include penicillin, streptomycin, puromycin, neomycin, hygromycin, and any combination thereof. In a preferred embodiment, a re-aggregation medium comprises penicillin and streptomycin.

In some embodiments, a re-aggregation medium herein can have one or more deoxyribonuclease, such as DNase I. In some embodiments, a re-aggregation medium herein can have a deoxyribonuclease in a concentration of about 0.1 mg/mL to about 10 mg/mL (e.g., about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL). In a preferred embodiment, a re-aggregation medium comprises about 1 mg/mL DNase I.

In some embodiments, a re-aggregation medium herein can have one or more apoptosis inhibitors. Non-limiting examples of apoptosis inhibitors include caspase inhibitors phosphatase inhibitors, PKC activator, nectrostatin inhibitor, adenosine receptor antagonist, cAMP phosphodiesterase inhibitor, autophagy inhibitor, superoxide dismutase mimetic, and the like. In some embodiments, the apoptosis inhibitor prevents mitochondrial outer membrane potential (MOMP) and/or mitochondrial fission, and/or protects the cells from mitochondrial apoptosis inducing factor (AIF) release and/or caspase-independent cell death. In some embodiments, the apoptosis inhibitor BI-6C9. In a preferred embodiment, a re-aggregation medium comprises about 10 μM BI-6C9.

In some embodiments, re-aggregation medium compositions can be prepared in a concentrated form. In some embodiments, re-aggregation medium compositions can be prepared in a concentrated form suitable for dilution. In some embodiments, re-aggregation medium compositions prepared in a concentrated form can be suitable for at least about 1×, about 2×, about 3×, about 4×, about 5×, about 6×, about 7×, about 8×, about 9×, or about 10× dilution. In some embodiments, diluted re-aggregation medium compositions can be used to wash cells before cryopreservation or after cryopreservation.

In certain embodiments, the present disclosure provides systems for use in the cryopreservation of one or more cell therapies. In some embodiments, a system for use in the cryopreservation of cell therapies can include a re-aggregation medium for the thawing or resizing cell therapies for use after cryopreservation.

In some embodiments the present disclosure provides a re-aggregation medium for use in thawing cells. For example, the first day after thaw, the first media comprises components MCDB 131, Glutamax, P/S (penicillin—streptomycin), Bovine Serum Albumin (BSA), Glucose, ZnSO4, an enzyme for digesting DNA (e.g., DNASE1), and/or apoptosis inhibitor (e.g., BI-6C9). The second media (e.g., which can be used any time after the first media, e.g., day 2) comprises components MCDB 131, Glutamax, P/S, BSA, Glucose, and/or ZnSO4.

Media components can be in quantities or concentrations that result in improved cell survival and cluster formation after thaw compared to traditional thawing media and methods.

In some embodiments, components may be added in a concentration of about 1 mg/mL. For example, components may be added in a concentration between about 0.1 mg/mL and about 100 mg/mL. For example, components may be added in a concentration of 0.1 mg/mL; about 0.2 mg/mL; about 0.3 mg/mL; about 0.4 mg/mL; about 0.5 mg/mL; about 0.6 mg/mL; about 0.7 mg/mL; about 0.8 mg/mL; about 0.9 mg/mL; about 1 mg/mL; about 1.1 mg/mL; about 1.2 mg/mL; about 1.3 mg/mL; about 1.4 mg/mL; about 1.5 mg/mL; about 1.6 mg/mL; about 1.7 mg/mL; about 1.8 mg/mL; about 1.9 mg/mL; about 2 mg/mL; about 2.1 mg/mL; about 2.2 mg/mL; about 2.3 mg/mL; about 2.4 mg/mL; about 2.5 mg/mL; about 2.6 mg/mL; about 2.7 mg/mL; about 2.8 mg/mL; about 2.9 mg/mL; about 3 mg/mL; about 3.1 mg/mL; about 3.2 mg/mL; about 3.3 mg/mL; about 3.4 mg/mL; about 3.5 mg/mL; about 3.6 mg/mL; about 3.7 mg/mL; about 3.8 mg/mL; about 3.9 mg/mL; about 4 mg/mL; about 4.1 mg/mL; about 4.2 mg/mL; about 4.3 mg/mL; about 4.4 mg/mL; about 4.5 mg/mL; about 4.6 mg/mL; about 4.7 mg/mL; about 4.8 mg/mL; about 4.9 mg/mL; about 5 mg/mL; about 5.1 mg/mL; about 5.2 mg/mL; about 5.3 mg/mL; about 5.4 mg/mL; about 5.5 mg/mL; about 5.6 mg/mL; about 5.7 mg/mL; about 5.8 mg/mL; about 5.9 mg/mL; about 6 mg/mL; about 6.1 mg/mL; about 6.2 mg/mL; about 6.3 mg/mL; about 6.4 mg/mL; about 6.5 mg/mL; about 6.6 mg/mL; about 6.7 mg/mL; about 6.8 mg/mL; about 6.9 mg/mL; about 7 mg/mL; about 7.1 mg/mL; about 7.2 mg/mL; about 7.3 mg/mL; about 7.4 mg/mL; about 7.5 mg/mL; about 7.6 mg/mL; about 7.7 mg/mL; about 7.8 mg/mL; about 7.9 mg/mL; about 8 mg/mL; about 8.1 mg/mL; about 8.2 mg/mL; about 8.3 mg/mL; about 8.4 mg/mL; about 8.5 mg/mL; about 8.6 mg/mL; about 8.7 mg/mL; about 8.8 mg/mL; about 8.9 mg/mL; about 9 mg/mL; about 9.1 mg/mL; about 9.2 mg/mL; about 9.3 mg/mL; about 9.4 mg/mL; about 9.5 mg/mL; about 9.6 mg/mL; about 9.7 mg/mL; about 9.8 mg/mL; about 9.9 mg/mL; about 10 mg/mL; about 10.1 mg/mL; about 10.2 mg/mL; about 10.3 mg/mL; about 10.4 mg/mL; about 10.5 mg/mL; about 10.6 mg/mL; about 10.7 mg/mL; about 10.8 mg/mL; about 10.9 mg/mL; about 11 mg/mL; about 11.1 mg/mL; about 11.2 mg/mL; about 11.3 mg/mL; about 11.4 mg/mL; about 11.5 mg/mL; about 11.6 mg/mL; about 11.7 mg/mL; about 11.8 mg/mL; about 11.9 mg/mL; about 12 mg/mL; about 12.1 mg/mL; about 12.2 mg/mL; about 12.3 mg/mL; about 12.4 mg/mL; about 12.5 mg/mL; about 12.6 mg/mL; about 12.7 mg/mL; about 12.8 mg/mL; about 12.9 mg/mL; about 13 mg/mL; about 13.1 mg/mL; about 13.2 mg/mL; about 13.3 mg/mL; about 13.4 mg/mL; about 13.5 mg/mL; about 13.6 mg/mL; about 13.7 mg/mL; about 13.8 mg/mL; about 13.9 mg/mL; about 14 mg/mL; about 14.1 mg/mL; about 14.2 mg/mL; about 14.3 mg/mL; about 14.4 mg/mL; about 14.5 mg/mL; about 14.6 mg/mL; about 14.7 mg/mL; about 14.8 mg/mL; about 14.9 mg/mL; about 15 mg/mL; about 15.1 mg/mL; about 15.2 mg/mL; about 15.3 mg/mL; about 15.4 mg/mL; about 15.5 mg/mL; about 15.6 mg/mL; about 15.7 mg/mL; about 15.8 mg/mL; about 15.9 mg/mL; about 16 mg/mL; about 16.1 mg/mL; about 16.2 mg/mL; about 16.3 mg/mL; about 16.4 mg/mL; about 16.5 mg/mL; about 16.6 mg/mL; about 16.7 mg/mL; about 16.8 mg/mL; about 16.9 mg/mL; about 17 mg/mL; about 17.1 mg/mL; about 17.2 mg/mL; about 17.3 mg/mL; about 17.4 mg/mL; about 17.5 mg/mL; about 17.6 mg/mL; about 17.7 mg/mL; about 17.8 mg/mL; about 17.9 mg/mL; about 18 mg/mL; about 18.1 mg/mL; about 18.2 mg/mL; about 18.3 mg/mL; about 18.4 mg/mL; about 18.5 mg/mL; about 18.6 mg/mL; about 18.7 mg/mL; about 18.8 mg/mL; about 18.9 mg/mL; about 19 mg/mL; about 19.1 mg/mL; about 19.2 mg/mL; about 19.3 mg/mL; about 19.4 mg/mL; about 19.5 mg/mL; about 19.6 mg/mL; about 19.7 mg/mL; about 19.8 mg/mL; about 19.9 mg/mL; or about 20 mg/mL.

In some embodiments, components may be added in a concentration of about 10 μM. For example, components may be added in a concentration of between about 0.01 μM and about 100 μM. For example, components may be added in a concentration of about 0.01 μM; about 0.02 μM; about 0.03 μM; about 0.04 μM; about 0.05 μM; about 0.06 μM; about 0.07 μM; about 0.08 μM; about 0.09 μM; about 0.1 μM; about 0.2 μM; about 0.3 μM; about 0.4 μM; about 0.5 μM; about 0.6 μM; about 0.7 μM; about 0.8 μM; about 0.9 μM; about 1 μM; about 1.5 μM; about 2 μM; about 2.5 μM; about 3 μM; about 3.5 μM; about 4 μM; about 4.5 μM; about 5 μM; about 5.5 μM; about 6 μM; about 6.5 μM; about 7 μM; about 7.5 μM; about 8 μM; about 8.5 μM; about 9 μM; about 9.5 μM; about 10 μM; about 10.5 μM; about 11 μM; about 11.5 μM; about 12 μM; about 12.5 μM; about 13 μM; about 13.5 μM; about 14 μM; about 14.5 μM; about 15 μM; about 15.5 μM; about 16 μM; about 16.5 μM; about 17 μM; about 17.5 μM; about 18 μM; about 18.5 μM; about 19 μM; about 19.5 μM; about 20 μM; about 20.5 μM; about 21 μM; about 21.5 μM; about 22 μM; about 22.5 μM; about 23 μM; about 23.5 μM; about 24 μM; about 24.5 μM; about 25 μM; about 25.5 μM; about 26 μM; about 26.5 μM; about 27 μM; about 27.5 μM; about 28 μM; about 28.5 μM; about 29 μM; about 29.5 μM; about 30 μM; about 30.5 μM; about 31 μM; about 31.5 μM; about 32 μM; about 32.5 μM; about 33 μM; about 33.5 μM; about 34 μM; about 34.5 μM; about 35 μM; about 35.5 μM; about 36 μM; about 36.5 μM; about 37 μM; about 37.5 μM; about 38 μM; about 38.5 μM; about 39 μM; about 39.5 μM; about 40 μM; about 40.5 μM; about 41 μM; about 41.5 μM; about 42 μM; about 42.5 μM; about 43 μM; about 43.5 μM; about 44 μM; about 44.5 μM; about 45 μM; about 45.5 μM; about 46 μM; about 46.5 μM; about 47 μM; about 47.5 μM; about 48 μM; about 48.5 μM; about 49 μM; about 49.5 μM; about 50 μM; about 50.5 μM; about 51 μM; about 51.5 μM; about 52 μM; about 52.5 μM; about 53 μM; about 53.5 μM; about 54 μM; about 54.5 μM; about 55 μM; about 55.5 μM; about 56 μM; about 56.5 μM; about 57 μM; about 57.5 μM; about 58 μM; about 58.5 μM; about 59 μM; about 59.5 μM; about 60 μM; about 60.5 μM; about 61 μM; about 61.5 μM; about 62 μM; about 62.5 μM; about 63 μM; about 63.5 μM; about 64 μM; about 64.5 μM; about 65 μM; about 65.5 μM; about 66 μM; about 66.5 μM; about 67 μM; about 67.5 μM; about 68 μM; about 68.5 μM; about 69 μM; about 69.5 μM; about 70 μM; about 70.5 μM; about 71 μM; about 71.5 μM; about 72 μM; about 72.5 μM; about 73 μM; about 73.5 μM; about 74 μM; about 74.5 μM; about 75 μM; about 75.5 μM; about 76 μM; about 76.5 μM; about 77 μM; about 77.5 μM; about 78 μM; about 78.5 μM; about 79 μM; about 79.5 μM; about 80 μM; about 80.5 μM; about 81 μM; about 81.5 μM; about 82 μM; about 82.5 μM; about 83 μM; about 83.5 μM; about 84 μM; about 84.5 μM; about 85 μM; about 85.5 μM; about 86 μM; about 86.5 μM; about 87 μM; about 87.5 μM; about 88 μM; about 88.5 μM; about 89 μM; about 89.5 μM; about 90 μM; about 90.5 μM; about 91 μM; about 91.5 μM; about 92 μM; about 92.5 μM; about 93 μM; about 93.5 μM; about 94 μM; about 94.5 μM; about 95 μM; about 95.5 μM; about 96 μM; about 96.5 μM; about 97 μM; about 97.5 μM; about 98 μM; about 98.5 μM; about 99 μM; about 99.5 μM; or about 100 μM.

In some embodiments, components may be added in a volume of about 5, about 478.8, or about 500 mL. For example, components may be added in a volume of between about 1 mL and about 1000 mL. For example, components may be added in a volume of about 1 mL; about 10 mL; about 20 mL; about 30 mL; about 40 mL; about 50 mL; about 60 mL; about 70 mL; about 80 mL; about 90 mL; about 100 mL; about 110 mL; about 120 mL; about 130 mL; about 140 mL; about 150 mL; about 160 mL; about 170 mL; about 180 mL; about 190 mL; about 200 mL; about 210 mL; about 220 mL; about 230 mL; about 240 mL; about 250 mL; about 260 mL; about 270 mL; about 280 mL; about 290 mL; about 300 mL; about 310 mL; about 320 mL; about 330 mL; about 340 mL; about 350 mL; about 360 mL; about 370 mL; about 380 mL; about 390 mL; about 400 mL; about 410 mL; about 420 mL; about 430 mL; about 440 mL; about 450 mL; about 460 mL; about 470 mL; about 480 mL; about 490 mL; about 500 mL; about 510 mL; about 520 mL; about 530 mL; about 540 mL; about 550 mL; about 560 mL; about 570 mL; about 580 mL; about 590 mL; about 600 mL; about 610 mL; about 620 mL; about 630 mL; about 640 mL; about 650 mL; about 660 mL; about 670 mL; about 680 mL; about 690 mL; about 700 mL; about 710 mL; about 720 mL; about 730 mL; about 740 mL; about 750 mL; about 760 mL; about 770 mL; about 780 mL; about 790 mL; about 800 mL; about 810 mL; about 820 mL; about 830 mL; about 840 mL; about 850 mL; about 860 mL; about 870 mL; about 880 mL; about 890 mL; about 900 mL; about 910 mL; about 920 mL; about 930 mL; about 940 mL; about 950 mL; about 960 mL; about 970 mL; about 980 mL; about 990 mL; or about 1000 mL.

In some embodiments, components may be added in a volume of about 523 μL. For example, components may be added in a volume between about 1 μL and 1000 μL. about 1 μL; about 10 μL; about 20 μL; about 30 μL; about 40 μL; about 50 μL; about 60 μL; about 70 μL; about 80 μL; about 90 μL; about 100 μL; about 110 μL; about 120 μL; about 130 μL; about 140 μL; about 150 μL; about 160 μL; about 170 μL; about 180 μL; about 190 μL; about 200 μL; about 210 μL; about 220 μL; about 230 μL; about 240 μL; about 250 μL; about 260 μL; about 270 μL; about 280 μL; about 290 μL; about 300 μL; about 310 μL; about 320 μL; about 330 μL; about 340 μL; about 350 μL; about 360 μL; about 370 μL; about 380 μL; about 390 μL; about 400 μL; about 410 μL; about 420 μL; about 430 μL; about 440 μL; about 450 μL; about 460 μL; about 470 μL; about 480 μL; about 490 μL; about 500 μL; about 510 μL; about 520 μL; about 530 μL; about 540 μL; about 550 μL; about 560 μL; about 570 μL; about 580 μL; about 590 μL; about 600 μL; about 610 μL; about 620 μL; about 630 μL; about 640 μL; about 650 μL; about 660 μL; about 670 μL; about 680 μL; about 690 μL; about 700 μL; about 710 μL; about 720 μL; about 730 μL; about 740 μL; about 750 μL; about 760 μL; about 770 μL; about 780 μL; about 790 μL; about 800 μL; about 810 μL; about 820 μL; about 830 μL; about 840 μL; about 850 μL; about 860 μL; about 870 μL; about 880 μL; about 890 μL; about 900 μL; about 910 μL; about 920 μL; about 930 μL; about 940 μL; about 950 μL; about 960 μL; about 970 μL; about 980 μL; about 990 μL; or about 1000 μL.

In some embodiments, components may be added in a weight or mass of about 0.23 g or about 10 g. For example, components may be added in a weight or mass of between about 0.01 g and 100 g. For example, components may be added at a weight or mass of 0.01 g; about 0.02 g; about 0.03 g; about 0.04 g; about 0.05 g; about 0.06 g; about 0.07 g; about 0.08 g; about 0.09 g; about 0.1 g; about 0.2 g; about 0.3 g; about 0.4 g; about 0.5 g; about 0.6 g; about 0.7 g; about 0.8 g; about 0.9 g; about 1 g; about 1.5 g; about 2 g; about 2.5 g; about 3 g; about 3.5 g; about 4 g; about 4.5 g; about 5 g; about 5.5 g; about 6 g; about 6.5 g; about 7 g; about 7.5 g; about 8 g; about 8.5 g; about 9 g; about 9.5 g; about 10 g; about 10.5 g; about 11 g; about 11.5 g; about 12 g; about 12.5 g; about 13 g; about 13.5 g; about 14 g; about 14.5 g; about 15 g; about 15.5 g; about 16 g; about 16.5 g; about 17 g; about 17.5 g; about 18 g; about 18.5 g; about 19 g; about 19.5 g; about 20 g; about 20.5 g; about 21 g; about 21.5 g; about 22 g; about 22.5 g; about 23 g; about 23.5 g; about 24 g; about 24.5 g; about 25 g; about 25.5 g; about 26 g; about 26.5 g; about 27 g; about 27.5 g; about 28 g; about 28.5 g; about 29 g; about 29.5 g; about 30 g; about 30.5 g; about 31 g; about 31.5 g; about 32 g; about 32.5 g; about 33 g; about 33.5 g; about 34 g; about 34.5 g; about 35 g; about 35.5 g; about 36 g; about 36.5 g; about 37 g; about 37.5 g; about 38 g; about 38.5 g; about 39 g; about 39.5 g; about 40 g; about 40.5 g; about 41 g; about 41.5 g; about 42 g; about 42.5 g; about 43 g; about 43.5 g; about 44 g; about 44.5 g; about 45 g; about 45.5 g; about 46 g; about 46.5 g; about 47 g; about 47.5 g; about 48 g; about 48.5 g; about 49 g; about 49.5 g; about 50 g; about 50.5 g; about 51 g; about 51.5 g; about 52 g; about 52.5 g; about 53 g; about 53.5 g; about 54 g; about 54.5 g; about 55 g; about 55.5 g; about 56 g; about 56.5 g; about 57 g; about 57.5 g; about 58 g; about 58.5 g; about 59 g; about 59.5 g; about 60 g; about 60.5 g; about 61 g; about 61.5 g; about 62 g; about 62.5 g; about 63 g; about 63.5 g; about 64 g; about 64.5 g; about 65 g; about 65.5 g; about 66 g; about 66.5 g; about 67 g; about 67.5 g; about 68 g; about 68.5 g; about 69 g; about 69.5 g; about 70 g; about 70.5 g; about 71 g; about 71.5 g; about 72 g; about 72.5 g; about 73 g; about 73.5 g; about 74 g; about 74.5 g; about 75 g; about 75.5 g; about 76 g; about 76.5 g; about 77 g; about 77.5 g; about 78 g; about 78.5 g; about 79 g; about 79.5 g; about 80 g; about 80.5 g; about 81 g; about 81.5 g; about 82 g; about 82.5 g; about 83 g; about 83.5 g; about 84 g; about 84.5 g; about 85 g; about 85.5 g; about 86 g; about 86.5 g; about 87 g; about 87.5 g; about 88 g; about 88.5 g; about 89 g; about 89.5 g; about 90 g; about 90.5 g; about 91 g; about 91.5 g; about 92 g; about 92.5 g; about 93 g; about 93.5 g; about 94 g; about 94.5 g; about 95 g; about 95.5 g; about 96 g; about 96.5 g; about 97 g; about 97.5 g; about 98 g; about 98.5 g; about 99 g; about 99.5 g; or about 100 g.

In some embodiments, components may be diluted prior to addition to the media. For example, the components may be diluted between about 1:1000 to about 1:4000. For example, the components may be diluted at about 1:1; about 1:100; about 1:200; about 1:300; about 1:400; about 1:500; about 1:600; about 1:700; about 1:800; about 1:900; about 1:1000; about 1:1100; about 1:1200; about 1:1300; about 1:1400; about 1:1500; about 1:1600; about 1:1700; about 1:1800; about 1:1900; about 1:2000; about 1:2100; about 1:2200; about 1:2300; about 1:2400; about 1:2500; about 1:2600; about 1:2700; about 1:2800; about 1:2900; about 1:3000%; 3100; about 1:3200; about 1:3300; about 1:3400%; 3500; about 1:3600; about 1:3700; about 1:3800; about 1:3900; about 1:4000; about 1:4100; about 1:4200; about 1:4300; about 1:4400; about 1:4500; about 1:4600; about 1:4700; about 1:4800; about 1:4900; about 1:5000; about 1:5100; about 1:5200; about 1:5300; about 1:5400; about 1:5500; about 1:5600; about 1:5700; about 1:5800; about 1:5900; or about 1:6000.

In some embodiments, the re-aggregation medium formulation comprises (e.g., for day 0) one or more components selected from: MCDB 131 (e.g., about 478.8 mL); Glutamax (e.g., about 5 mL); P/S (e.g., about 5 mL); BSA (e.g., about 10 g) Glucose (e.g., about 0.23 g); ZnSO4 (e.g., about 523 μL); DNASE1 (an enzyme for digesting DNA) (e.g., about 1 mg/mL of a 1:1000 dilution); and/or BI-6C9 (an apoptosis inhibitor) (e.g., about 10 μM at a 1:4000 dilution). In some embodiments, the re-aggregation medium formulation comprises (e.g., for day 2 and on) one or more components selected from: MCDB 131 (e.g., about 478.8 mL); Glutamax (e.g., about 5 mL); P/S (e.g., about 5 mL); BSA (e.g., about 10 g); Glucose (e.g., about 0.23 g); and/or ZnSO4 (e.g., about 523 μL).

In some embodiments, the re-aggregation medium comprises, consists essentially of or consists of a basal medium with trace elements, putrescine, adenine, thymidine, sodium bicarbonate, L-alanyl-L-glutamine, penicillin, streptomycin, bovine serum albumin, glucose, ZnSO₄ and optionally DNase I and BI-6C9.

In some embodiments, the re-aggregation medium comprises, consists essentially of or consists of a basal medium with trace elements, putrescine, adenine, thymidine, sodium bicarbonate, about 2 mM L-alanyl-L-glutamine, penicillin, streptomycin, about 300 μM bovine serum albumin, about 0.115 g/L glucose, ZnSO₄ and optionally about 1 mg/mL DNase I and about 10 μM BI-6C9.

### II. Methods

In an aspect of the present disclosure provides methods of thawing, resizing, and/or culturing cell therapies (e.g. pluripotent stem cell-derived islets (SC-islets)). In some embodiments, methods of the present disclosure include a method of preparing human pluripotent stem cell-derived islets (SC-islets) comprising: (i) thawing cryopreserved SC-islets and incubating in a first re-aggregation medium of the disclosure (e.g. MCDB 131, Glutamax, P/S, BSA, Glucose, ZnSO4, DNASE1, and/or BI-6C9) for about 24 hours; and/or (ii) incubating the SC-islets of (i) in a second re-aggregation medium of the disclosure (e.g. MCDB 131, Glutamax, P/S, BSA, Glucose, and/or ZnSO4). Suitable formulations of the re-aggregation medium are describe in Section I and incorporated into this section by reference. In some embodiments, the method further comprises aggregating SC islets. In some embodiments, the method further comprises resizing the thawed SC-islets. In some embodiments, the method further comprises maturing the thawed SC-islets. In some embodiments, components are in an amount to result in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw. In some embodiments, the thawed SC-islets are incubated for a period of time sufficient to result in: high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw. In some embodiments, incubating thawed SC-islets in one or both of the formulations results in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

In some embodiments, the cell therapies are contacted with a first re-aggregation medium from about 6 hours to about 30 hours. In certain embodiments, the cell therapies are contacted with a first re-aggregation medium for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours. In an exemplary embodiment, the cell therapies are contacted with a first re-aggregation medium for about 24 hours.

In some embodiments, cell therapies can be maintained in re-aggregation medium of the disclosure for a time sufficient for maintenance, passage, or to resize SC-islets. In some embodiments, the cell therapy is cultured for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days. In some embodiments, the cell therapies can be maintained in re-aggregation medium of the disclosure from between 1-5 days, 1-10 days, 1-15 days, 1-20 days, 5-10 days, 5-15 days, 5-20 days, 10-15 days, 10-20 days, or 15-20 days. In some embodiments, cell therapies can be maintained in re-aggregation medium of the disclosure for at least about 5 days (e.g., about 10 days). In some embodiments, the re-aggregation medium is replenished as required during this time.

Following cryopreservation, frozen cell therapies can be thawed in accordance with the methods described below or known in the art.

Frozen cells are preferably thawed quickly (e.g., in a water bath maintained at 37°−41° C.) and chilled immediately upon thawing. In a specific embodiment, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting SC-islets capable or beta cells to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogenic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include and can comprise, but are not limited to cell media, MCDB 131, Glutamax, P/S, BSA, Glucose, ZnSO4, enzyme for digesting DNA, such as DNASE1, an apoptosis inhibitor, such as BI-6C9, or combinations thereof. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, the term “consisting essentially of” takes it's well established meaning and is generally construed to mean that the composition or formulation (a) necessarily includes the listed ingredients and (b) is open to unlisted ingredients that do not materially affect the basic and novel properties of the composition (e.g. resulting in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw relative to standard techniques).

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Re-Aggregation Media for Cryopreserved SC-Islet

There is a problem of thawing cryopreserved SC-islets leads to significant cell death after several days in culture. This resulted in unfavorable cluster morphologies leading to poor islet health and function. This invention addresses this problem. Successful thawing of SC-islets improves their utility for diabetes cell replacement therapy.

Resizing of SC-islets leads to improvements in SC-islets. However, this results in the death of some cells. This invention addresses this problem. Improving SC-islets increases their utility for diabetes cell replacement therapy.

Maturation of SC-islets produced in vitro is still a limitation for the utility of these cells in cell replacement therapy. This invention improves the maturation of the cells, increasing their utility for diabetes cell replacement therapy.

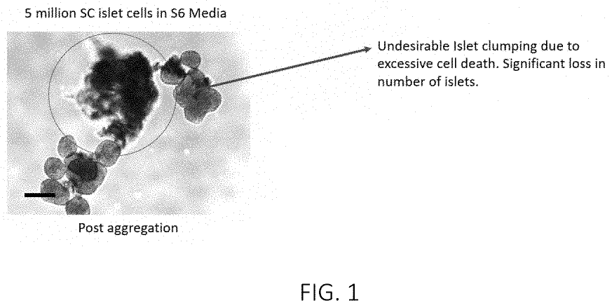

Conventional media causes undesirable cell clumping after thaw, see e.g. FIG. 1, 5 million SC islet cells in S6 media post aggregation.

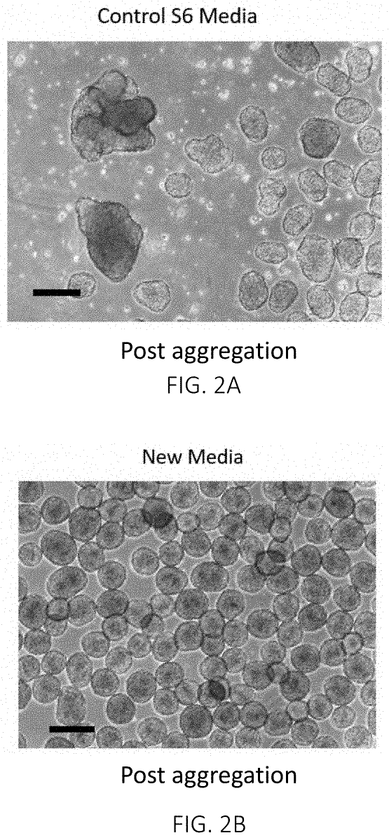

However, using new formulations as shown in Table 1 and Table 2, recovery and clustering morphology is significantly improved, see e.g. FIG. 2A-2B.

TABLE 1
Re-aggregation medium first day after thaw (Day 0)

| | | |
|---|---|---|
| Component | Amount | Dilution |

| | | | |
|---|---|---|---|
| MCDB 131 | 478.8 | mL | |
| Glutamax | 5 | mL | |
| P/S | 5 | mL | |
| BSA | 10 | g | |
| Glucose | 0.23 | g | |
| ZnSO4 | 523 | uL | |
| DNASE1 | 1 | mg/mL | 1:1000 |
| BI-6C9 | 10 | uM | 1:4000 |

TABLE 2
Re-aggregation medium Day 2 onwards

| | | |
|---|---|---|
| Component | Amount | Dilution |

| | | |
|---|---|---|
| MCDB 131 | 478.8 | mL |
| Glutamax | 5 | mL |
| P/S | 5 | mL |
| BSA | 10 | g |
| Glucose | 0.23 | g |
| ZnSO4 | 523 | uL |

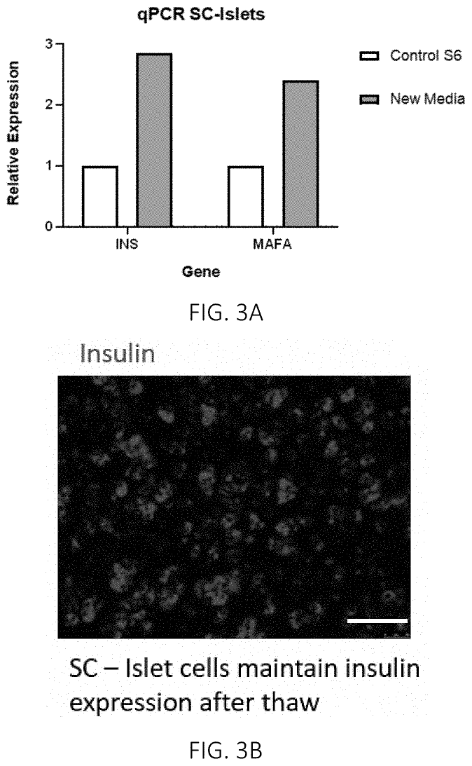

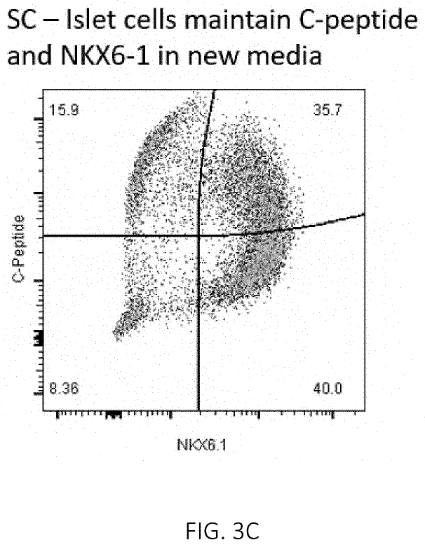

Re-aggregation medium shows improved gene expression for SC Islets and maintains Beta cell identity, see e.g. FIG. 3A-3C.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, a “population” of cells refers to a group of at least 2 cells, e.g. 2 cells, 3 cells, 4 cells, 10 cells, 100 cells, 1000 cells, 10,000 cells, 100,000 cells or any value in between, or more cells. Optionally, a population of cells can be cells which have a common origin, e.g. they can be descended from the same parental cell, they can be clonal, they can be isolated from or descended from cells isolated from the same tissue, or they can be isolated from or descended from cells isolated from the same tissue sample. Preferably, the population of hematopoietic progenitor cells is substantially purified. As used herein, the term “substantially purified” means a population of cells substantially homogeneous for a particular marker or combination of markers. By substantially homogeneous is meant at least 80%, at least 85%, at least 90%, at least 95%, at least 96%, at least 97%, at least 98%, at least 99% or more homogeneous for a particular marker or combination of markers.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The terms “composition” and “formulation” are used interchangeably.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs); and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is a non-human animal. In certain embodiments, the animal is a fish or reptile.

The term “administer,” “administering,” or “administration” refers to implanting, absorbing, ingesting, injecting, inhaling, or otherwise introducing a compound or cell described herein or generated as described herein, or a composition thereof, in or on a subject.

The terms “treatment,” “treat,” and “treating” refer to reversing, alleviating, delaying the onset of, or inhibiting the progress of a disease. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen and/or in light of detecting that the subject has a genotype associated with the disease). Treatment may also be continued after symptoms have resolved, for example, to delay or prevent recurrence.

The terms “condition,” “disease,” and “disorder” are used interchangeably.

The term “stem cell” refers to a vertebrate cell that has the ability both to self-renew, and to generate differentiated progeny. The ability to generate differentiated progeny may be described as pluripotent (see Morrison et al. (1997) Cell 88:287-298). “Embryonic stem cells” (ES cells) are pluripotent stem cells derived from the inner cell mass of a blastocyst, an early-stage preimplantation embryo. Pluripotency distinguishes embryonic stem cells from adult stem cells found in adults; while embryonic stem cells can generate all cell types in the body, adult stem cells are multipotent and can produce only a limited number of cell types.

“Induced pluripotent stem cells”, abbreviated as iPS cells, are a type of pluripotent stem cell artificially derived from a non-pluripotent cell, typically an adult somatic cell, by inducing expression of certain genes {e.g., injection of an expression construct). Induced pluripotent stem cells are identical in many respects to natural pluripotent stem cells, such as embryonic stem (ES) cells {e.g., in their physical properties). They may be the same in their expressions of certain stem cell genes and proteins, chromatin methylation patterns, doubling time, embryoid body formation, teratoma formation, viable chimera formation, and potency and differentiability. The term “induced pluripotent stem cell” encompasses pluripotent cells, that, like embryonic stem (ES) cells, can be cultured over a long period of time while maintaining the ability to differentiate into all types of cells in an organism. However, unlike ES cells (which are typically derived from the inner cell mass of blastocysts), iPS cells are derived from differentiated somatic cells, that is, cells that have a narrower, more defined potential.

By “culturing” the cell means growing the cells in an artificial, in vitro environment. By “maintaining” means continuing to grow the cells in culture under suitable conditions until the pluripotency state of the cell is converted to a more naïve state.

“Cell line” refers to a population of largely or substantially identical cells, wherein the cells have often been derived from a single ancestor cell or from a defined and/or substantially identical population of ancestor cells. For example, a cell line may consist of descendants of a single cell. A cell line may have been or may be capable of being maintained in culture for an extended period (e.g., months, years, for an unlimited period of time). It will be appreciated that cells may acquire mutations and possibly epigenetic changes over time such that some individual cells of a cell line may differ with respect to each other. In some embodiments, at least 80%, 85%, 86%, 87%, 88%, 89%, 90%, 91%, 92%, 93%, 94%, 95%, 96%, 97%, 98%, or 99% of the cells of a cell line or cell culture are at least 95%, 96%, 97%, 98%, or 99% genetically identical. In some embodiments, at least 80%, 85%, 86%, 87%, 88%, 89%, 90%, 91%, 92%, 93%, 94%, 95%, 96%, 97%, 98%, or 99% of the cells of a cell line or cell culture express the same set of cell surface markers. The set of markers could be markers indicative of ground state (naïve) pluripotency or cell-type specific markers.

A “clone” refers to a cell derived from a single cell without change. It will be understood that if cells of a clone are subjected to different culture conditions or if some of the cells are subjected to genetic modification, the resulting cells may be considered distinct clones.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of thawing, resizing, maintaining, or passaging at least one SC-islet cell, the method comprising:
culturing the at least one SC-islet cell in the presence of basal medium, at least one glutamine alternative, at least one antibiotic, one or more albumin, at least one energy substrate, at least one trace element, and optionally one or more deoxyribonuclease and at least one apoptosis inhibitor.

2. The method of claim 1, wherein the basal medium is MCDB 131.

3. The method of claim 1, wherein the at least one glutamine alternative is L-alanyl-L-glutamine dipeptide.

4. The method of claim 1, wherein the at least one antibiotic is penicillin and streptomycin.

5. The method of claim 1, wherein the one or more albumin is bovine serum albumin.

6. The method of claim 1, wherein the at least one energy substrate is derived from a carbohydrate.

7. The method of claim 6, wherein the energy substrate is glucose.

8. The method of claim 1, wherein the at least one trace element is ZnSO₄.

9. The method of claim 1, wherein the one or more deoxyribonuclease is DNase I.

10. The method of claim 1, wherein the apoptosis inhibitor is BI-6C9.

11. The method of claim 1, wherein the SC-islet cell were cryopreserved and thawed by contacting the SC-islet cell with a first re-aggregation medium comprising MCDB 131, L-alanyl-L-glutamine dipeptide, penicillin streptomycin, bovine serum albumin, glucose, ZnSO₄, DNase I, and BI-6C9 for about 24 hours.

12. The method of claim 11, wherein the method further comprises contacting the SC-islet cell with a second re-aggregation medium comprising MCDB 131, L-alanyl-L-glutamine dipeptide, penicillin streptomycin, bovine serum albumin, glucose, and ZnSO₄.

13. The method of claim 1, wherein contacting the SC-islet with medium results in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

14. The method of claim 1, wherein the thawed SC-islets are incubated for a period of time sufficient to result in: high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

15. The method of claim 12, wherein contacting the thawed SC-islets in one or both of the formulations results in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw.

16. A cell culture medium, wherein the cell culture medium is the first re-aggregation medium of claim 11.

17. A cell culture medium, wherein the cell culture medium is the second re-aggregation medium of claim 12.

18. A kit for preparing the cell culture medium of claim 16, wherein the kit comprises individually packaged components, basal medium, and instructions for preparing the cell culture medium.

19. A kit for preparing the cell culture medium of claim 17, wherein the kit comprises individually packaged components, basal medium, and instructions for preparing the cell culture medium.
